(19) United States
(12) Patent Application Publication
(10) Pub. No.: US 2014/0221982 A1
(43) Pub. Date: Aug. 7, 2014

(54) SEMIPERMEABLE MEDICAL POUCHES AND THEIR USES

(71) Applicant: Erik Gatenholm, Blacksburg, VA (US)

(72) Inventor: Erik Gatenholm, Blacksburg, VA (US)

(21) Appl. No.: 14/014,030

(22) Filed: Aug. 29, 2013

Related U.S. Application Data
(60) Provisional application No. 61/694,662, filed on Aug. 29, 2012.

(51) Int. Cl. A61M 37/00; A61L 31/04; A61F 2/02

(52) U.S. Cl. CPC A61M 37/00; A61F 2/022; A61L 31/042
USPC 604/522; 264/240; 264/28; 604/93.01

(57) ABSTRACT

A novel process for bacterial production of nanocellulose hydrogel pouches for applications for cell therapy, cell encapsulation, cell delivery, cell proliferation and cell differentiation has been invented. The process is based on fermentation of bacteria producing nanocellulose in oxygen permeable mold interconnected with tubing with diameter less than 5 mm. The resulting Bacterial Nanocellulose (BNC) hydrogel pouch is biocompatible and non-degradable in the human body. The inner wall of the pouch is highly porous and supports cell migration, cell proliferation and cell differentiation. The wall of the pouch allows controlled diffusion of extracellular components. The BNC pouch with injected cells can be implanted and used for treatment of diseases such as diabetes by local delivery of insulin. There are numerous applications of BNC pouches with injected cells in tissue engineering, regenerative medicine and cancer treatment.

### CROSS REFERENCE TO RELATED APPLICATIONS

This application claims priority to and the benefit of the filing date of U.S. Provisional Application No. 61/694,662, filed Aug. 29, 2012, the disclosure of which is hereby incorporated by reference herein in its entirety.

### BACKGROUND OF THE INVENTION

1. Field of the Invention

The present innovation relates to the field of medical devices, tissue engineering, regenerative medicine, and cell therapy, more particularly, to systems and methods for production by bacteria of nanocellulose medical devices in the form of pouches with tubing which allow cell injection and media exchange.

2. Description of Related Art

Regenerative strategies using autologous or stem cells for many diseases are limited by poor cellular survival, distribution and integration after transplantation into the host body. What is needed are suitable biomaterials which can act as compartment-packaging and a delivery system at the same time. In addition, such packaging should be very functional and act rather as a bioreactor for the selected cells. It should allow cells to migrate, proliferate and differentiate. It should keep nutrients which cells need but allow waste products of cell metabolism to be released. It should also allow oxygen transport into the bioreactor and carbon dioxide out of the bioreactor. If cells are indented to be used for cell therapy, the bioreactor should allow diffusion of components such as insulin (or other drugs or substances) out from bioreactor. If the cells from another patient or from another species (xenotransplantation) are used the bioreactor should have the ability to provide immunoisolation.

The current art of biomaterials provides shaped cellulose tubes (hollow) with limited thickness, which have been produced in the method using tubular bioreactor as described in WO2001061026. Oxygen delivery through the silicon support has been explored for manufacturing of tubes for applications such as vascular grafts as described in EP2079845 and WO2008040729 A2.

This invention describes an ultimate solution for all these challenges, specifically, a nanocellulose pouch grown by bacteria in a silicone template. This unique biomaterial behaves like hydrogel and shows diffusion properties which qualify it for the mentioned purposes. At the same time the pouch has good mechanical properties for long term storage of cells.

### SUMMARY OF THE INVENTION

A biofabrication process is provided in which bacteria suspension in media is injected into a gas permeable pouch mold with attached tubing and then cultivated for 1-5 days. The process involves growth of pouches in molds with tubing with diameter smaller than 5 mm. The preferential material used for the mold is an oxygen permeable thin silicone mold in the shape of a pouch. In this process the bacteria suspension in growth medium is injected into the silicone mold and the nanocellulose pouch is produced by the bacteria during the 1-5 days.

The hydrogel-like pouches, after removal of bacteria by washing with alkali, and then with de-ionized water can then be processed in different ways. One way is to dehydrate the pouch by freeze drying or solvent exchange and then introduce another component in water solution such as a growth factor, another polymer, drugs, conductive polymers or antibacterial agents. If desired, then cells can be injected. In embodiments, the added component, such as cells, is able to slow release out of the BNC Pouch. In embodiments, the pouch walls are porous to allow diffusion of certain sized molecules yet containment of larger sized molecules inside or outside the pouch. The outer side of the pouch is smooth and allows good tissue integration whereas the inner wall(s) of the pouch are more open and allow cells to grow. The pouch can be used as a bioreactor for cell proliferation and/or cell differentiation. Typically, the BC pouches of the invention are not degraded in the human body and thus are well suited as cell delivery and cell containment devices. An ideal application is to use BNC as a container for transplantation of transdifferentiated cells derived from the patient's own differentiated cells, such as for example, the use of insulin-producing beta cells transdifferentiated from isolated hepatocytes as a treatment for diabetes. Since the BNC biomaterial has unique barrier properties the pouch acts also as an immunoisolation device. The pouches can thus be used for xenotransplantation of non-human cells that are used to produce a needed substance. For example, treating diabetic patients by introducing insulin-producing pig cells directly into their muscle.

Biosynthetic nanocellulose, a natural polysaccharide, is an attractive biomaterial because of its good mechanical properties, hydroexpansivity, biocompatibility and its stability within a wide range of temperatures and pH levels. Cellulose ((β-1→4-glucan) is the most abundant polymer of natural origin. In addition to being biosynthesized in vast amounts as structural material in the walls of plants, cellulose is also produced as an exopolysaccharide, i.e., bacterial nanocellulose (BNC), by Gluconacetobacter xylinus. Biomaterial applications require pure material and often an introduction of functional groups to stimulate the tissue regeneration process. BNC has additional advantages as a biomaterial as compared to plant-derived cellulose. Apart from good mechanical strength, high water holding capacity, high purity and accessibility to non-aggregated micro fibrils, the BNC can be molded into desirable shapes for a given application, allowing one to produce a three dimensional network of micro fibrils.

It has been previously described that it is advantageous to cultivate bacteria inside permeable tubes. Current innovation describes cultivation of bacteria in a silicone mold in the form of pouch. Bacteria produce hydrogel-like material with smooth outer shell and porous inner wall. Such hydrogel, after removal of bacteria, form a perfect bioreactor for cell growth and cell therapy. The bacterial nanocellulose is biocompatible and integrated by tissue. It allows vascularization and is not degraded in human body. It allows small molecules to diffuse through the pouch wall while keeping cells inside.

Particular aspects of the invention include a process to produce robust Bacterial Nanocellulose, BNC pouches with interconnected tubing with control of size, diameter, morphology, porosity and mechanical properties consisting of two steps: (a) growth of BNC hydrogel in oxygen permeable mold in the form of pouch with tubing diameter less than 5 mm by injecting into the tubing the suspension of cellulose producing bacteria in suitable medium, and (b) modifying the density of wall of the pouch by freezing and freeze drying process followed by rewetting. Processes of the invention in particular include combining a BNC hydrogel pouch with silicone tubing for cell injection.

Such BNC hydrogel pouches produced by such methods can be seeded with cells and used for cell delivery and cell therapy.

Other applications include where a BNC hydrogel pouch is used as a container for transplantation of transdifferentiated cells derived from the patient's own differentiated cells.

Additional applications include using a BNC hydrogel pouch for transplantation of insulin-producing beta cells transdifferentiated from isolated hepatocytes as a treatment for diabetes. Even further, the BNC hydrogel pouches can be used for xenotransplantation of non-human cells for treating diabetic patients.

Even further, applications for the BNC hydrogel pouches can include use of the pouches for tissue engineering, regenerative medicine and/or cancer treatment.

In particular, embodiments of the invention include a process of preparing Bacterial Nanocellulose (BNC) pouches comprising one or more or all of (in any order): providing an oxygen permeable mold in the form of a pouch; disposing a growth medium suitable for growing BNC in the mold; disposing one end of a 5-mm or less diameter tubing in the growth medium; injecting cellulose producing bacteria through the tubing into the growth medium; and growing BNC hydrogel in the mold in a manner sufficient to form of a BNC pouch.

Such processes can comprise modifying wall density of the pouch by any one or more of (in any combination) freezing, freeze drying, and/or rewetting the pouch.

Such pouches can be seeded with cells.

Processes of the invention include administering the pouch to a subject in a manner to provide for cell delivery and cell therapy to the subject.

Embodiments further provide a BNC hydrogel pouch comprising silicone tubing disposed in the pouch in a manner to provide for injection of cells into the pouch through the tubing. Such pouches can comprise cells seeded onto the pouch.

Processes of the invention also include a process of administering cell delivery and cell therapy to a subject comprising administering such pouches to the subject.

A process of transplanting cells to a subject is also included which comprises disposing transdifferentiated cells derived from the subject's own differentiated cells in the pouch and administering the pouch to the subject. When referring to “pouch” or “method” or “process” in the context of this invention, it is important to note that any pouch, method, or process described in this specification or figures can be used for that particular embodiment. Indeed, any pouch, method, or process or part thereof described in this specification and figures can be used in any other pouch, method, or process also described in this specification and figures in any manner.

Even further, embodiments include a process of transplanting cells to a subject comprising disposing insulin-producing beta cells transdifferentiated from isolated hepatocytes in the pouch and administering the pouch to the subject in a manner to treat diabetes.

Such processes of the invention include a process of xenotransplantation comprising disposing non-human cells in a pouch of the invention and administering the pouch to a diabetic patient.

### BRIEF DESCRIPTION OF THE DRAWINGS

The accompanying drawings illustrate certain aspects of embodiments of the present invention, and should not be used to limit or define the invention. Together with the written description the drawings serve to explain certain principles of the invention.

FIG. 1 is an image of a template used for production of a silicone mold used for the biofabrication production system.

FIG. 2 is an image of the medium with bacteria in the silicone mold after 2 days of cultivation in which the BNC hydrogel has been already formed.

FIG. 3 is an image of BNC pouches which have been removed from silicone molds and are under purification process in alkali followed by rinsing with DI water.

FIG. 4 is an image of a purified BNC pouch with inserted silicone tubing for use in cell therapy.

### DETAILED DESCRIPTION OF VARIOUS EMBODIMENTS OF THE INVENTION

Reference will now be made in detail to various exemplary embodiments of the invention. Embodiments described in the description and shown in the figures are illustrative only and are not intended to limit the scope of the invention. Changes may be made in the specific embodiments described in this specification and accompanying drawings that a person of ordinary skill in the art will recognize are within the scope and spirit of the invention.

Biosynthetic nanocellulose, such as that produced by the bacteria Gluconacetobacter xylinus, is an emerging biomaterial with great potential to be used for cell delivery and cell therapy. It has excellent mechanical properties and it is biocompatible. It is also attractive for cell immobilization and cell support since cells grow very well onto nanocellulose. It is composed of nanocellulose fibrils which bind water and thus behaves as a hydrogel. The transport properties of this biomaterial can be modified. A novel method has been invented here to grow BNC hydrogel in the form of a pouch with tubing with controlled length and diameter. Such BNC pouches can be loaded with cells and implanted into body.

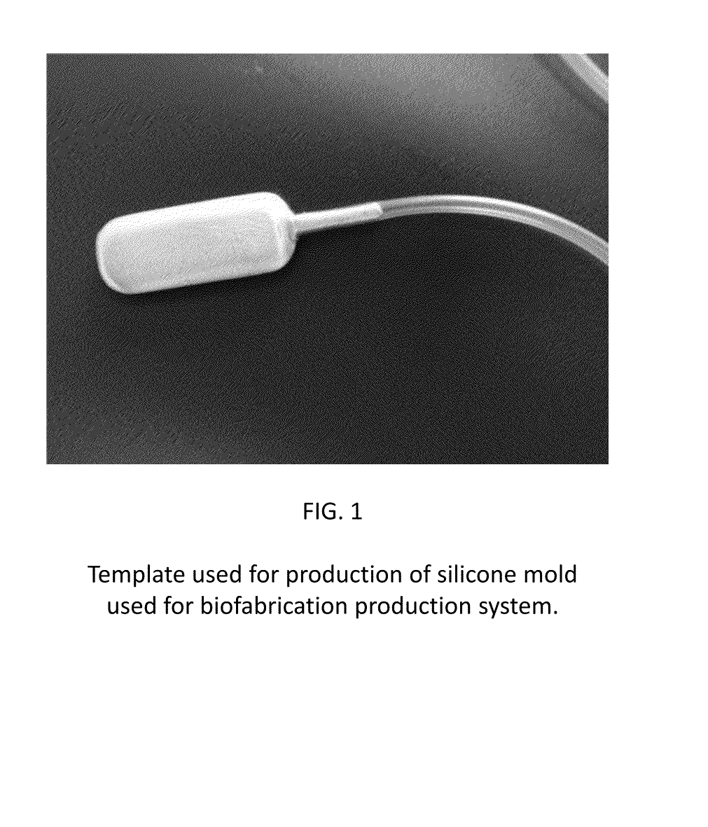

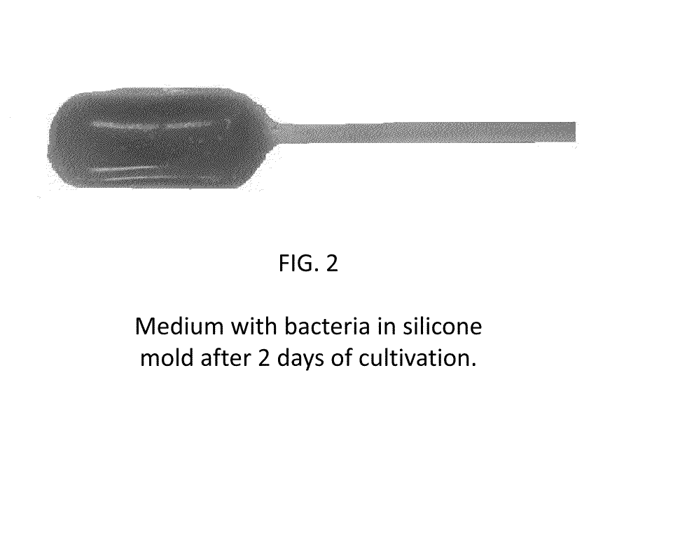

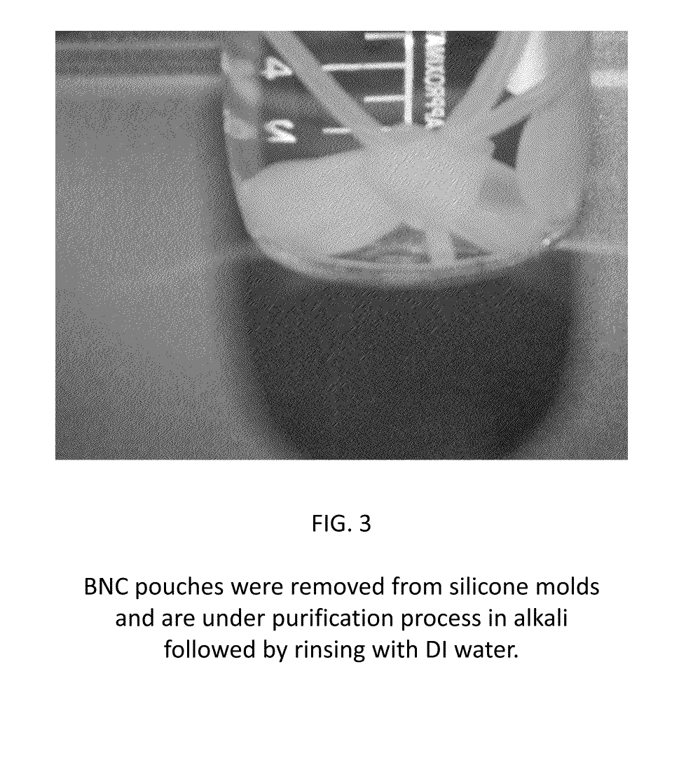

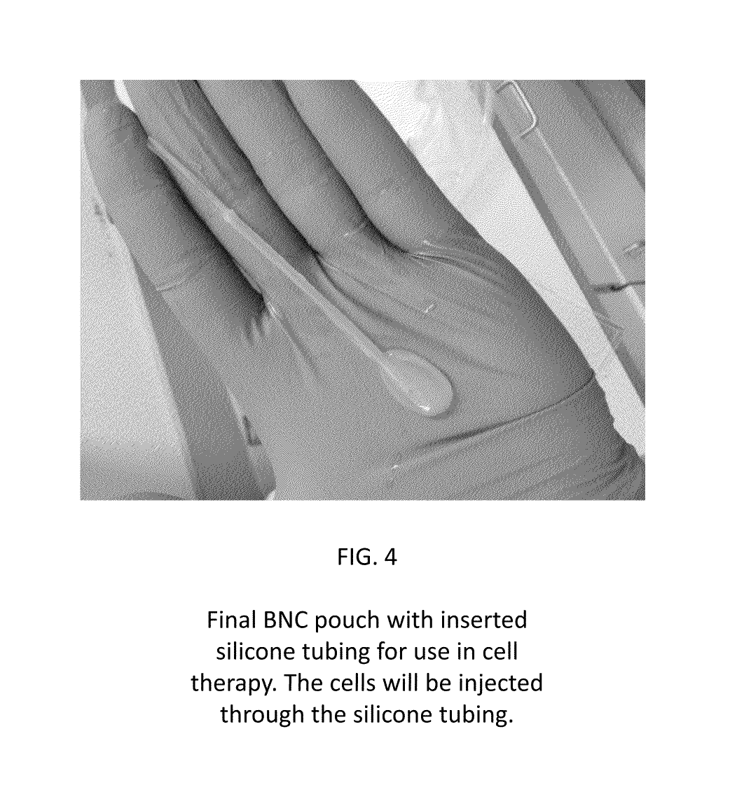

The process starts with production of a silicone mold with well defined dimensions (see FIG. 1) according to a particular desired application. Bacteria is suspended in a suitable medium and is injected into the silicone mold. The mold with bacteria suspension is placed in an incubator holding at about 30 degree C. After about 24 hours the BNC hydrogel is formed at the wall of the silicone mold and inside the tubing (see FIG. 2). The growth process can continue between 1 and 5 days depending on the diameter of the silicone tubing. The environment around the silicone tubing can be enriched in oxygen in order to increase production of cellulose in the mold but humidity is also desired in order to avoid drying of the solution in the tubing. After cultivation is terminated the silicone mold is cut open and the BNC hydrogel is removed for treatment in a purification step (see FIG. 3). First the pouches are washed in 0.1 N NaOH solution for 24 hours at room temperature and then rinsed with copious amount of de-ionized water. After the purification process the pouches can be equipped with silicone tubing to enable easy handling of cell injection and medium addition for cell growth (FIG. 4).

### EXAMPLES

### Example 1

### Biofabrication of BNC Pouch

BNC pouches have been grown in a silicon mold produced using the template shown in FIG. 1. The system is composed of a mold containing sterilized silicone. The silicone pouch (PDMS) is produced in house, with tubing with internal diameter of 2 mm, as was used in this example. 10 ml of medium composed of (fructose [4% w/v], yeast extract [0.5% w/v], (NH4)2SO4 [0.33% w/v], KH2PO4 [0.1% w/v], MgSO4.7H2O [0.025% w/v], corn steep liquor [2% v/v], trace metal solution [1% v/v, (30 mg EDTA, 14.7 mg CaCl2.2H2O, 3.6 mg FeSO4.7H2O, 2.42 mg Na2MoO4.2H2O, 1.73 mg ZnSO4.7H2O, 1.39 mg MnSO4.5H2O and 0.05 mg CuSO4.5H2O in 1 liter distilled water)] and vitamin solution [1% v/v (2 mg inositol, 0.4 mg pyridoxine HCl, 0.4 mg niacin, 0.4 mg thiamine HCl, 0.2 mg para-aminobenzoic acid, 0.2 mg D-panthothenic acid calcium, 0.2 mg riboflavin, 0.0002 mg folic acid and 0.0002 mg D-biotin in 1 liter distilled water)]) was sterilized by microfiltration and placed in the sterile syringe. 0.5 ml of Gluconacetobacter xylinus subsp.sucrofermentas BPR2001, trade number 700178™, from the ATCC bacteria suspension taken from preculture was added to 10 ml of medium. The bacteria suspension was injected into the silicone pouches placed in an incubator holding 30 degree Celsius for 2 days. The time can be varied between 1 and 5 days. In embodiments, the most important factor is to not fill the tubing by BC hydrogel. It is preferential to keep the temperature of medium at 30 degree C. The production of cellulose starts at the inner surface of the silicone mold and proceeds towards the opposite end of the tubing. BC layers form most readily at the intersection of the solid, liquid, air boundary. In some experiments oxygen gas (still keeping high moisture content) can be added for example outside the silicone tubing to increase density of produced cellulose.

### Example 2

### Conversion of BNC Hydrogel into Robust Cell Delivery and Cell Therapy Device

The BNC hydrogel pouch produced in Example 1 was removed from the silicone mold by cutting open the mold (FIG. 2). The BNC pouch is then washed with 0.1 M alkali solution for 24 hours at 25 degree Celsius and then washed several times with distilled water (see FIG. 3). After the washing process the pouch has been extracted and extract was analyzed regarding endotoxin content using LAL test. The endotoxin level was lower than 0.5 EU/ml. The diffusion properties of the pouch can be modified by freezing the pouch followed by freeze drying and rewetting.

### Example 3

### Use of BNC Pouch for Cell Therapy

The BNC pouch produced in Example 1 and converted into a robust cell delivery and cell therapy device has been loaded with different cells. Human Mesenchymal Stem Cells were used to study cell proliferation and differentiation. Cells show good survival in the BNC pouch and were able to migrate, proliferate and differentiate when differentiation medium was added. As shown in FIG. 4 the final BNC pouch has inserted silicone tubing for use in cell therapy. The cells can be injected through the silicone tubing.

The present invention has been described with reference to particular embodiments having various features. In light of the disclosure provided, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A process of preparing Bacterial Nanocellulose (BNC) pouches comprising:
providing an oxygen permeable mold in the form of a pouch;
disposing a growth medium suitable for growing BNC in the mold;
disposing one end of a 5-mm or less diameter tubing in the growth medium;
injecting cellulose producing bacteria through the tubing into the growth medium; and
growing BNC hydrogel in the mold in a manner sufficient to form of a BNC pouch.

2. The process of claim 1 comprising modifying wall density of the pouch by freezing, freeze drying, and rewetting the pouch.

3. The process of claim 2 comprising seeding the pouch with cells.

4. The process of claim 3 comprising administering the pouch to a subject in a manner to provide for cell delivery and cell therapy to the subject.

5. A BNC hydrogel pouch comprising silicone tubing disposed in the pouch in a manner to provide for injection of cells into the pouch through the tubing.

6. The pouch of claim 5, wherein the pouch comprises cells seeded onto the pouch.

7. A process of administering cell delivery and cell therapy to a subject comprising administering the pouch of claim 6 to the subject.

8. A process of transplanting cells to a subject comprising disposing transdifferentiated cells derived from the subject's own differentiated cells in the pouch of claim 5 and administering the pouch to the subject.

9. A process of transplanting cells to a subject comprising disposing insulin-producing beta cells transdifferentiated from isolated hepatocytes in the pouch of claim 5 and administering the pouch to the subject in a manner to treat diabetes.

10. A process of xenotransplantation comprising disposing non-human cells in the pouch of claim 5 and administering the pouch to a diabetic patient.
